(19) United States
(12) Patent Application Publication
(10) Pub. No.: US 2023/0117531 A1
(43) Pub. Date: Apr. 20, 2023

(54) DRIED PARTICLE INHALATION FOR DELIVERY OF CANNABIS

(71) Applicant: TFF Pharmaceuticals, Inc., Fort Worth, TX (US)

(72) Inventors: Glenn MATTES, Fort Worth, TX (US); Aaron FLETCHER, Fort Worth, TX (US); Carlo CASULO, Fort Worth, TX (US)

(73) Assignee: TFF Pharmaceuticals, Inc., Fort Worth, TX (US)

(21) Appl. No.: 17/906,529

(22) PCT Filed: Mar. 18, 2021
(86) PCT No.: PCT/US2021/022868

§ 371 Date: Sep. 16, 2022

Related U.S. Application Data
(60) Provisional application No. 62/991,896, filed on Mar. 19, 2020.

(51) Int. Cl. A61K 9/00; A61K 31/352; A61K 31/05; A61K 9/19

(52) U.S. Cl. CPC A61K 9/0075; A61K 31/352; A61K 31/05; A61K 9/19

(57) ABSTRACT

The disclosure relates to dry powder compositions of (a) a plant-based cannabidiol (pCBD) composition, or (b) a plant-based tetrahydrocannabinol composition (pTHC); or (c) a synthetic cannabidiol composition (sCBD), or (d) a synthetic tetrahydrocannabinol composition (sTHC), or (e) a plant-based cannabidiol (pCBD) composition in combination with a a plant-based tetrahydrocannabinol composition (pTHC), or (f) a synthetic cannabidiol composition (sCBD) in combination with a synthetic tetrahydrocannabinol composition (sTHC) and their delivery to the airway of a subject using a dry powder inhaler.

This application claims the benefit of U.S. Provisional Patent Application No. 62/991,896, filed Mar. 19, 2020, the entirety of which is incorporated herein by reference.

### BACKGROUND

### I. Technical Field

The present disclosure relates to dry powder inhalers for local or systemic delivery of an active ingredient to and/or through the lungs. The inhalers are used with inhalable dry powders, including primarily medicament formulations comprising an active cannabis agent for the treatment of various diseases and disorders.

### II. Related Art

Drug delivery to lung tissue has been achieved using a variety of devices for inhalation, including nebulizers and inhalers, such as metered dose inhalers and dry powder inhalers to treat local disease or disorders. Dry powder inhalers used to deliver medicaments to the lungs contain a dose system of a powder formulation usually either in bulk supply or quantified into individual doses stored in unit dose compartments, like hard gelatin capsules or blister packs. Bulk containers are equipped with a measuring system operated by the patient in order to isolate a single dose from the powder immediately before inhalation.

Dosing reproducibility with inhalers requires that the drug formulation is uniform and that the dose be delivered to a subject with consistency and reproducible results. Therefore, the dosing system ideally should operate to completely discharge all of the formulation effectively during an inspiratory maneuver when the patient is taking his/her dose. However, complete powder discharge from the inhaler is not required as long as reproducible dosing can be achieved.

Dry powder inhalers can be breath activated or breath-powered and can deliver drugs by converting drug particles in a carrier into a fine dry powder which is entrained into an air flow and inhaled by the patient. Drugs delivered with the use of a dry powder inhaler for local lung delivery include those used to treat allergy, asthma and/or chronic obstructive pulmonary disease (COPD). Dry powder inhalers are, however, no longer only intended to treat pulmonary disease but can also be used to treat systemic disease as the drug is still delivered to the lungs but is absorbed into the systemic circulation.

### SUMMARY

The present disclosure concerns dry powders and dry powder inhalers comprising a dry powder containing cannabis materials. In some aspects, these powders may be used for inhalation for delivery to the lungs for local or systemic delivery into the pulmonary circulation. In some aspects, the dry powder inhaler comprises a dry powder composition of: (a) a plant-based cannabidiol (pCBD) composition; (b) a plant-based tetrahydrocannabinol composition (pTHC); (c) a synthetic cannabidiol composition (sCBD); (d) a synthetic tetrahydrocannabinol composition (sTHC); (e) a plant-based cannabidiol (pCBD) composition in combination with a plant-based tetrahydrocannabinol composition (pTHC); or (f) a synthetic cannabidiol composition (sCBD) in combination with a synthetic tetrahydrocannabinol composition (sTHC). In certain aspects, a dry powder of the embodiments is produced by thin film freezing (TFF), see, e.g., US20100221343, which is incorporated herein by reference. In some aspects, pCBD, pTHC, sCBD and/or sTHC compositions are formed as brittle matrix particles for use in a dry powder inhaler (see, e.g., US20180147161, which is incorporated herein by reference).

Also provided is a method of delivering a dry powder composition of (a) a plant-based cannabidiol (pCBD) composition; (b) a plant-based tetrahydrocannabinol composition (pTHC), (c) a synthetic cannabidiol composition (sCBD); (d) a synthetic tetrahydrocannabinol composition (sTHC); (e) a plant-based cannabidiol (pCBD) composition in combination with a plant-based tetrahydrocannabinol composition (pTHC); or (f) a synthetic cannabidiol composition (sCBD) in combination with a synthetic tetrahydrocannabinol composition (sTHC) to the airway of a subject comprising administering said dry powder composition to an airway of said subject with a dry powder inhaler. In certain aspects, a dry powder of the embodiments is produced by TFF.

In a further embodiment, a dry powder or dry powder inhaler comprises a dry powder composition of (a) a plant-based cannabidiol (pCBD) composition in combination with a plant-based tetrahydrocannabinol composition (pTHC), or (b) a synthetic cannabidiol composition (sCBD) in combination with a synthetic tetrahydrocannabinol composition (sTHC). The dry powder composition comprises a pCBD:pTHC in a ratio of 10:1, 5:1, 2:1, 1:1, 1:2, 1:5 or 1:10, or a sCBD:sTHC in a ratio of 10:1, 5:1, 2:1, 1:1, 1:2, 1:5 or 1:10.

Also provided is a method of delivering a dry powder composition of (a) a plant-based cannabidiol (pCBD) composition in combination with a plant-based tetrahydrocannabinol composition (pTHC); or (b) a synthetic cannabidiol composition (sCBD) in combination with a synthetic tetrahydrocannabinol composition (sTHC) to the airway of a subject comprising administering said dry powder composition to an airway of said subject with a dry powder inhaler.

The dry powder inhaler may be a breath-powered inhaler, is compact, may be reusable or disposable, may be various shapes and sizes, and comprises a system of airflow conduit pathways for the effective and rapid delivery of powder medicament to the lungs and the systemic circulation.

In one embodiment, the thy powder inhaler comprises a unit dose cartridge, and a dry powder formulation that is to be aerosolized and delivered to lung tissue for a local tissue effect, or for absorption into the blood stream in the lungs and be delivered by the systemic circulation to target tissue or organs of a subject. In an embodiment, the dry powder can comprise, a carrier molecule, including pharmaceutically acceptable carriers and excipients, for example, phospholipids, polymers such as polyethylene glycol, co-glycolides, a saccharide, a polysaccharide, or a diketopiperazine, and an active ingredient.

The dry powder may comprise an inhalable dry powder, including a pharmaceutical formulation comprising a cannabis agent for pulmonary delivery. In some embodiments, delivery is to the deep lung (that is, to the alveolar region) and in some of these embodiments, the active agent or active ingredient is absorbed into the pulmonary circulation for systemic targeted or general use.

Cartridges for use with the dry powder inhaler can be manufactured to contain the dry powder medicament for inhalation. In one embodiment, the cartridge is structurally configured to be adaptable to a particular dry powder inhaler and can be made of any size and shape, depending on the size and shape of the inhaler to be used with, for example, if the inhaler has a mechanism which allows for translational movement or for rotational movement.

In some embodiments, the dry powder formulation is dispensed with consistency from the inhaler in less than about three (3) seconds, or generally less than one (1) second. In some embodiments, the inhaler air conduits are designed to yield high resistance to air flow values of, for example, approximately 0.065 to about 0.200 (kPa)/liter per minute. Therefore, in the inhalation system, peak inhalation pressure drops of between 2 and 20 kPa produce resultant peak flow rates of about between 7 and 70 liters per minute. These flow rates result in greater than 75% of the cartridge contents dispensed in fill masses between 1 and 50 mg. In some embodiments, these performance characteristics are achieved by end users within a single inhalation maneuver to produce cartridge dispense percentage of greater than 90%. In certain embodiments, the inhaler and cartridge system are configured to provide a single dose by discharging powder from the inhaler s a continuous flow, or as one or more pulses of powder delivered to a patient.

One of ordinary skill in the art will appreciate that starting materials, biological materials, reagents, synthetic methods, purification methods, analytical methods, assay methods, and biological methods other than those specifically exemplified can be employed in the practice of the disclosure without resort to undue experimentation. All art-known functional equivalents, of any such materials and methods are intended to be included in this disclosure.

The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure claimed.

Thus, in some aspects, the present disclosure is directed to dry powder inhalers comprising a dry powder containing cannabis materials for inhalation for delivery to the lungs for local or systemic delivery into the pulmonary circulation.

The dry powder inhaler may be a breath-powered inhaler, is compact, may be reusable or disposable, may be various shapes and sizes, and comprises a system of airflow conduit pathways for the effective and rapid delivery of powder medicament to the lungs and the systemic circulation.

Thus, in some embodiments a pCBD, pTHC, sCBD, and/or sTHC unit-dose delivery system is provided as a template for use in a dry powder inhaler. In some aspects, a unit-dose delivery system comprising one or more concave indentations; a cover positioned to sealed the one or more concave indentations; and a brittle matrix medicinal formulation appropriate for pulmonary delivery in at least one of the one or more concave indentations, wherein the brittle matrix medicinal formulation comprises a non-tightly packed porous flocculated web matrix comprising one or more brittle-matrix particles of one or more active agents, wherein a portion of the one or more brittle-matrix particles is delivered and templated by the formation of one or more particles upon atomization from the unit-dose delivery system using a dry powder inhaler to form a respirable porous particle for deep lung delivery.

In some aspects, a dry powder of the embodiments is produced by a method:

(A) admixing two or more pCBD, pTHC, sCBD, sTHC or mixture thereof into a solvent wherein the solvent comprises an organic solvent and water to form a pharmaceutical composition wherein the pharmaceutical composition (e.g., a composition that comprises an amount of the active agents in the solvent from about 0.01% to about 10% (w/v));

(B) applying the pharmaceutical composition to a rotating surface wherein the surface is at a temperature from about −70° C. to about −120° C.; and

(C) freezing the pharmaceutical composition to form a dry solid composition.

Thus, it should be understood that although the present disclosure has been specifically disclosed by particular embodiments and optional features, modification and variation of the concepts herein disclosed may be resorted to by those skilled in the art, and that such modifications and variations are considered to be within the scope of this disclosure as defined by the appended claims.

### DETAILED DESCRIPTION

In embodiments disclosed herein, dry powder inhalers and method of using the same are disclosure for the delivery of cannabis materials to a subject by oral inhalation. In particular, the materials include particular ratios of plant-based CBD and THC, or particular ratios of synthetic CBC and THC. Further details regarding the disclosure are set out below.

### I. Definitions

As used herein the term “a unit dose inhaler” refers to an inhaler that is adapted to receive a single cartridge or container comprising a dry powder formulation and delivers a single dose of a dry powder formulation by inhalation from a single container to a user. It should be understood that in some instances multiple unit doses will be required to provide a user with a specified dosage.

As used herein a “cartridge” is an enclosure configured to hold or contain a dry powder formulation, a powder containing enclosure, which has a cup or container and a lid. The cartridge is made of rigid materials, and the cup or container is moveable relative to the lid in a translational motion or vice versa.

As used herein a “powder mass” is referred to an agglomeration of powder particles or agglomerate having irregular geometries such as width, diameter, and length.

As used herein a “unit dose” refers to a pre-metered dry powder formulation for inhalation. Alternatively, a unit dose can be a single container having multiple doses of formulation that can be delivered by inhalation as metered single amounts. A unit dose cartridge/container contains a single dose. Alternatively, it can comprise multiple individually accessible compartments, each containing a unit dose.

As used herein, the term “about” is used to indicate that a value includes the standard deviation of error for the device or method being employed to determine the value.

As used herein, the term “microparticle” refers to a particle with a diameter of about 0.5 to about 1000 μm, irrespective of the precise exterior or interior structure. Microparticles having a diameter of between about 0.5 and about 10 microns can reach the lungs, successfully passing most of the natural barriers. A diameter of less than about 10 microns is required to navigate the turn of the throat and a diameter of about 0.5 μm or greater is required to avoid being exhaled. To reach the deep lung (or alveolar region) where most efficient absorption is believed to occur, it is preferred to maximize the proportion of particles contained in the “respirable fraction” (RF), generally accepted to be those particles with an aerodynamic diameter of about 0.5 to about 6 .mu.m, though some references use somewhat different ranges, as measured using standard techniques, for example, with an Anderson Cascade Impactor. Other impactors can be used to measure aerodynamic particle size such as the NEXT GENERATION IMPACTOR® (NGI®, MSP Corporation), for which the respirable fraction is defined by similar aerodynamic size, for example <6.4 μm. In some embodiments, a laser diffraction apparatus is used to determine particle size, for example, the laser diffraction apparatus disclosed in U.S. Pat. No. 8,508,732, which disclosure is incorporated herein in its entirety for its relevant teachings related to laser diffraction, wherein the volumetric median geometric diameter (VMGD) of the particles is measured to assess performance of the inhalation system. For example, in various embodiments cartridge emptying of ≥80%, 85%, or 90% and a IMGD of the emitted particles of <12.5 μm, <7.0 μm, or <4.8 μm can indicate progressively better aerodynamic performance.

Respirable fraction on fill (RF/fill) represents the percentage (%) of powder in a dose that is emitted from an inhaler upon discharge of the powder content filled for use as the dose, and that is suitable for respiration, i.e., the percent of particles from the filled dose that are emitted with sizes suitable for pulmonary delivery, which is a measure of microparticle aerodynamic performance. A RF/fill value of 40% or greater than 40% reflects acceptable aerodynamic performance characteristics. In certain embodiments disclosed herein, the respirable fraction on fill can be greater than 50%. In an exemplary embodiment, a respirable fraction on fill can be up to about 80%, wherein about 80% of the fill is emitted with particle sizes <5.8 μm as measured using standard techniques.

As used herein, the term “dry powder” refers to a fine particulate composition that is not suspended or dissolved in a propellant, or other liquid. It is not meant to necessarily imply a complete absence of all water molecules.

As used herein, “amorphous powder” refers to dry powders lacking a definite repeating form, shape, or structure, including all non-crystalline powders.

### II. TFF and Brittle Matrix Particles

In some aspects, the present disclosure provides the use of brittle matrix particles (BMP). The brittle matrix particles that may be used herein are characterized by their low density configuration with a high surface area and high porosity. These brittle matrix particles may be prepared using convention methods such as spray freeze drying or thin film freezing as described herein and in U.S. Patent Application No. 2010/0221343 and Watts, et al., 2013, both of which are incorporated herein by reference. Additionally, formulations of CBD have been prepared using similar methods in PCT/US2020/051388, which is incorporated herein by reference. In some embodiments, thin film freezing is used to prepare the brittle matrix particles described herein. After freezing, these particles may be further subjected to drying to obtain a dry powder suitable for aerosol administration. The brittle matrix particles may be dried through lyophilization and other methods known to those of skill in the art. Without wishing to be bound by any theory, the brittle matrix particles and the fast freezing drying methods allow the mixing of the particles while maintaining the homogeneity of the mixture wile preventing segregation of the different components. The improved homogeneity may also be exhibited during the aerosolization process.

In some aspects, the brittle matrix particles are prepared using thin film freezing (TFF) methods. Such preparation may be used in a manner to allow for the deposition of THC and/or CBD and one or more excipients to form a pharmaceutical composition. In some embodiments, the methods comprise dissolving the pharmaceutical composition in a solvent. Some solvents which may be used in the methods described herein include water, an organic solvent, or a mixture thereof. The organic solvents that may be used herein include polar organic solvents such an alcohol, a heterocyclic compound, an alkylnitrile, or a mixture thereof. Some non-limiting examples of polar organic solvents include methanol, ethanol, isopropanol, tert-butanol (tertiary butanol), dimethyl sulfoxide, dimethylformamide, 1,4-dioxane, or acetonitrile. In some aspects, mixtures of these solvents are contemplated. Such mixtures may comprise one or more organic solvents with water. One non-limiting example of these mixtures includes the solvent mixture of tert-butanol, 1,4-dioxane, acetonitrile, and water. The solvent mixture may comprise a mixture of tertiary butanol, 1,4-dioxane, acetonitrile, and purified water in a ratio of 2:1:3:3 (v/v).

In some aspects, the present disclosure comprises a combination of two or more active pharmaceutical ingredients (APIs) such as THC and/or CBD. These combinations may further comprises one or more excipients. Some non-limiting examples of some excipients which may be used herein include a sugar or sugar derivative, such as mannitol, trehalose, or lactose, or an amino acid, such as glycine. These compositions may be dissolved in a solvent as described herein.

In some aspects, the THC and/or CBD composition comprises an excipient. In other aspects, the active pharmaceutical ingredient is formulated in the pharmaceutical composition without an excipient. When the composition comprises an excipient, the excipient may be present from about no excipient to a molar ratio of about 1:9 active pharmaceutical ingredients to the excipient. In some embodiments, the molar ratio of active pharmaceutical ingredients to excipients is from about a composition comprising no excipient to a molar ratio comprising about 1:1 ratio of active pharmaceutical ingredients to excipients. The molar ratio of active pharmaceutical ingredients to excipients may be about 1:1.

The composition may be dissolved in a solvent as described above. When the composition is dissolved in a solvent, the total amount of the THC and/or CBD composition in the solvent may be from about 0.1% to about 10% (w/v). The total amount of the THC and/or CBD composition may be from about 0.1% to about 6% (w/v). In some aspects, the total amount of THC and/or CBD composition is less than 6%, 5%, 4%, 3.5%, 3%, 2.5%, 2.0%, 1.75%, 1.5%, 1 1.25%, 1.0%, 0.9%, 0.8%, 0.7%, 0.6%, 0.5%, 0.4%, 0.3%, 0.2%, or 0.1%, or any range derivable therein. The total amount of the THC and/or CBD composition in the solvent is preferable less than 6%, more preferably less than 5%. Using small amount of the THC and/or CBD composition in the solvent is believed to give the advantageous properties such as leading to the formation of a brittle matrix particle and thus using less than 6% (w/v) and preferably less than 5% (w/v) is recommended. While lower amounts of the compounds are beneficial, the concentrations below 0.01% (w/v) or more preferably 0.1% (w/v) may lead to solutions too dilute to obtain a useful pharmaceutical composition. In some embodiments, the total amount of the pharmaceutical composition is about 0.5% (w/v).

In some aspects, the compositions are prepared using a thin film apparatus. The apparatus may be used to apply the solution to a surface such as a stainless steel and then frozen. This surface may also be rotating such that without wishing to be bound by any theory, it is believed that the rotating prompts the even application of the solution to the surface. The solution may be frozen at a cryogenic temperature such as a temperature below −50° C. Cryogenic temperatures include a temperature form about −50° C. to about −270° C., form about −70° C. to about −120° C. or form about −75° C. to about −100° C. In some embodiments, the cryogenic temperature is about 90° C.±3° C. In some aspects, the samples are stored frozen. In other aspects, the samples are lyophilized to obtain a dry powder. Lyophilization is known to those of skill in the art and is taught in U.S. Pat. Nos. 5,756,468, 6,440,101, 8,579,855, and PCT Patent Application Publication No. WO 2009/125986, which are incorporated herein by reference. In some aspects, it may be advantageous to store the composition at room temperature. The lyophilized samples may be prepared such that the temperature is gradually increased from the lyophilization temperature of less than −40° C. to a temperature around room temperature such as about 25° C. Also, he increase in temperature may be carried out under a vacuum or in a reduced pressure environment and/or an environment which has a reduced moisture content such as a desiccator.

The present disclosure provides, in some aspects, brittle matrix particles which have a high surface area compared to other techniques such as jet milling or physical mixtures. In some aspects, the brittle matrix particles with two or more active pharmaceutical compositions have a specific surface area of greater than 5 m²/g. The brittle matrix particles may have a specific surface area from about 5 m²/g to about 1000 m²/g, from about 10 m ²/g to about 500 m²/g, or from about 20 m²/g to about 250 m²/g. In some embodiments, the specific surface area is from about 5, 6, 7, 8, 9, 10, 15, 20, 25, 30, 35, 40, 45, 50, 60, 70, 80, 90, 100, 125, 150, 175, 200, 225, 250, 300, 350, 400, 450, 500, 600, 700, 800, 900, to about 1000 m²/g, or any range derivable therein.

The brittle matrix particles comprising THC and/or CBD composition described herein may have a total emitted dose (or emitted dose) of greater than 80% of the active ingredient. The total emitted dose may also be from about 80% to about 100%, from about 85% to about 100%, or from about 90% to about 100%. The formulations of the pharmaceutical composition described herein may have an total emitted dose of greater than 80%, 81%, 82%, 83%, 84%, 85%, 86%, 87%, 88%, 89%, 90%, 91%, 97%, 93%, 94%, 95%, 96%, 97%, 98% or 99%, or any range derivable therein.

The pharmaceutical compositions described herein may comprise one or more excipients. Excipients are components which are not therapeutically active but may be used in the formation of a pharmaceutical composition. The excipients used herein include amino acids, sugars, sugar derivatives, or other excipients know those of skill in the art. In particular, the present disclosure includes the use of a sugar such as trehalose, lactose, glucose, fructose, or mannose, or a sugar derivative such as an aminosugar such as glucosamine or a sugar alcohol such as mannitol. Other excipients which may be used include amino acids such as alanine or glycine.

In some aspects, the brittle matrix particles component contains two or more THC or CBD components with one or more excipients to form a pharmaceutical composition. The pharmaceutical composition can thus be formulated in the brittle matrix particles in an amorphous form or in a particular crystalline form. In some embodiments, the pharmaceutical composition is formulated in the amorphous form. Additionally, the brittle matrix particles that may be used are a low density particle.

The present disclosure provides methods which makes use of the brittle matrix particles in the aerosol administration of a pharmaceutical composition. Without wishing to be bound by any theory, it is believed that the brittle matrix particles are readily fractured during the aerosolization thus enhancing the delivery of the pharmaceutical composition. The fracturing of the particles may be used to enhance the composition's ability to aerosolize and dispersion during administration.

III. Inhaler Devices

The dry powder inhalers disclosed herein may be of various shapes and sizes, and can be reusable, easy to use, inexpensive to manufacture and/or produced in high volumes in simple steps using plastics or other acceptable materials. Various embodiments of the dry powder inhalers are provided herein and in general, the inhalation systems comprise inhalers, powder-filled cartridges, and empty cartridges. The present inhalation systems can be designed to be used with any type of dry powder. In one embodiment, the dry powder is a relatively cohesive powder which requires optimal deagglomeration conditions.

Commercially available multi-dose inhalers such as FLOVENT® DISKUS, ADVAIR® DISKUS, and PULMICORT® FLEXHALER to name a few. For example, the AFREZZA® inhaler is a unit dose dry powder inhaler, which delivers a human insulin formulation for the treatment of diabetes in humans. AFREZZA was approved by the U.S. Food and Drug Administration for the treatment of diabetes type 1 and type 2 in June 2014. The AFREZZA inhaler is a breath-actuated, multiple use inhaler which delivers a single dose of insulin contained in a cartridge to the lungs, wherein the insulin is absorbed into the circulation for the effective treatment of hyperglycemia associated with diabetes.

Dry powder inhalers such as those described in U.S. Pat. Nos. 7,305,986, 7,464,706, 8,499,757, 8,636,001, and U.S. Patent Publication No. 20170216538, which disclosures are incorporated herein by reference in their entirety, can generate primary drug particles, or suitable inhalation plumes during an inspiratory maneuver by deagglomerating the powder formulation within a capsule or cartridge comprising a single dose. The amount of fine powder discharged from the inhaler's mouthpiece during inhalation is largely dependent on, for example, the inter-particulate forces in the powder formulation and the efficiency of the inhaler to separate those particles so that they are suitable for inhalation. The benefits of delivering drugs via the pulmonary circulation are numerous and include rapid entry into the arterial circulation, avoidance of drug degradation by liver metabolism, and ease of use without discomfort.

In exemplary embodiments herewith, the present devices can be manufactured by several methods and from various materials. In one embodiment, the inhalers and cartridges are made, for example, by injection molding techniques, thermoforming, blow molding, pressing, 3D printing, and the like using various types of plastic materials, including, polypropylene, cyclicolephin co-polymer, nylon, and other compatible polymers and the like. In certain embodiments, the dry powder inhaler can be assembled using top-down assembly of individual component parts. In some embodiments, the inhalers are generally provided in compact sizes, for example, from about 1 inch to about 5 inches in dimension, and generally, the width and height are less than the length of the device. In certain embodiments the inhaler is provided in various shapes including, relatively rectangular bodies, although other shapes can be used such as cylindrical, oval, tubular, squares, oblongs, and circular forms.

The inhalers effectively fluidize, deagglomerate or aerosolize a dry powder formulation by using at least one relatively rigid flow conduit pathway for allowing an airflow to enter the inhaler. For example, the inhaler is provided with a first air flow pathway for entering and exiting a cartridge containing the dry powder, and a second air pathway which can merge with the first air flow pathway exiting the cartridge. The flow conduits, for example, can have various shapes and sizes depending on the inhaler configuration. In one embodiment, inhalers are high resistance inhalers with resistance value of, for example, approximately 0.065 to about 0.200 (kPa)/liter per minute. Therefore, in the system, peak inhalation pressure drops of between 2 and 20 kPa produce resultant peak flow rates of about between 7 and 70 liters per minute. These flow rates result in greater than 75% of the cartridge contents dispensed in fill masses between 1 and 50 mg. In some embodiments, these performance characteristics are achieved by end users within a single inhalation maneuver to produce cartridge dispense percentage of greater than 90% of the powder contained in a cartridge.

Cartridge embodiments for use with the inhalers are described in U.S. Pat. No. 8,424,518, which disclosure is incorporated by reference in its entirety. In summary, a cartridge for use with the inhaler embodiments disclosed herewith comprises two parts, although other embodiments may be envisioned. The cartridges are configured to contain a dry powder medicament in a storage, tightly sealed or contained position and can be reconfigured within an inhaler from a powder containment position to an inhalation or dosing configuration. In certain embodiments, the cartridge comprises a lid and a cup having one or more apertures, a containment configuration and dosing configuration, an outer surface, an inner surface defining an internal volume; and the containment configuration restricts communication to the internal volume and the dispensing configuration forms an air passage through said internal volume to allow an air flow to enter and exit the internal volume in a predetermined manner. For example, the cartridge container can be configured so that an airflow entering the cartridge air inlet is directed across the air outlets within the internal volume to meter the medicament leaving the cartridge so that rate of discharge of a powder is controlled; and wherein airflow in the cartridge can tumble substantially perpendicular to the air outlet flow direction, mix and fluidize a powder in the internal volume prior to exiting through dispensing apertures. Cartridges for use with the instant inhalers can be provided in individual blisters or grouped in a blister depending in the need of the subject or the hygroscopicity of the formulation with respect to stability of powder and/or the active ingredient.

In embodiments, the dry powder inhaler and cartridge form an inhalation system which can be structurally configured to effectuate a tunable or modular airflow resistance, as it can be effectuated by varying the cross-sectional area or geometries of the air conduits at any section of the airflow pathway of the system. In one embodiment, the dry powder inhaler system geometries of the air conduits can generate an airflow resistance value of from about 0.065 to about 0.200 (kPa)/liter per minute. In other embodiments, a check valve may be employed to prevent air flow through the inhaler until a desired pressure drop, such as 4 kPa has been achieved, at which point the desired resistance reaches a value within the range given herewith.

In yet another embodiment, an inhalation system for delivering a dry powder formulation to a patient is provided. The system comprises an inhaler including a container mounting area configured to receive a container and a mouthpiece having at least two inlet apertures and at least one exit aperture; wherein one inlet aperture of the at least two inlet apertures is in communication with the container area, and one of the at least two inlet apertures is in communication with the at least one exit aperture via a flow path configured to bypass the container area to deliver the dry powder formulation to the patient; wherein the flow conduit configured to bypass the container area delivers 30% to 90% of the total flow going through the inhaler during an inhalation.

In another embodiment, a dry powder inhalation system for delivering a dry powder formulation to a patient is also provided. The system comprises a dry powder inhaler including a mounting and reconfiguring region for a cartridge; said dry powder inhaler and cartridge combined are configured to have at least two airflow pathways which are rigid flow conduits in a dosing configuration and a plurality of structural regions that provide a mechanism for powder deagglomeration of the inhalation system in use; wherein at least one of the plurality of mechanisms for deagglomeration is an agglomerate size exclusion aperture in the container region having a smallest dimension between 0.5 mm and 3 mm.

In embodiments disclosed herein, a dry powder formulation can consist of a crystalline powder, an amorphous powder, or combinations thereof, wherein the powder is dispensed with consistency from the inhaler in less than about 2 seconds. The present inhaler system has a high resistance value of approximately 0.065 to about 0.200 (kPa)/liter per minute. Therefore, in the system comprising a cartridge, peak inhalation pressure drops applied of between 2 and 20 kPa produce resultant peak flow rates of about through the system of between 7 and 70 liters per minute. These flow rates result in greater than 75% of the cartridge contents dispensed in fill masses between 1 and 30 mg, or up to 50 mg of powder. In some embodiments, these performance characteristics are achieved by end users within a single inhalation maneuver to produce cartridge dispense percentage of greater than 90%. In certain embodiments, the inhaler and cartridge system are configured to provide a single dose by discharging powder from the inhaler as a continuous flow, or as one or more pulses of powder delivered to a patient. In an embodiment, an inhalation system for delivering a dry powder formulation to a patient's lung(s) is provided, comprising a dry powder inhaler configured to have flow conduits with a total resistance to flow in a dosing configuration ranging in value from 0.065 to about 0.200 (kPa)/liter per minute. In this and other embodiments, the total resistance to flow of the inhalation system is relatively constant across a pressure differential range of between 0.5 kPa and 7 kPa.

The structural configuration of the inhaler can permit the deagglomeration mechanism to produce respirable fractions greater than 50% and particles of less than 5.8 μm. The inhalers can discharge greater than 85% of a powder medicament contained within a container during an inhalation maneuver. Generally, the inhalers herein depicted herewith can discharge greater that 90% of the cartridge contents or container contents in less than 3 seconds at pressure differentials between 2 and 5 kPa with fill masses ranging up to 30 mg or 50 mg.

While inhalers are primarily described as breath-powered, in some embodiments, the inhaler can be provided with a source for generating the pressure differential required to deagglomerate and deliver a dry powder formulation. For example, an inhaler can be adapted to a gas-powered source, such as compressed gas stored energy source, such as from a nitrogen can, which can be provided at the air inlet ports. A spacer can be provided to capture the plume so that the patient can inhale at a comfortable pace.

In embodiments, the inhaler can be provided as a reusable inhaler for delivering a single unit dose. A reusable inhaler means that it can be used multiple times which can be predetermined depending on the formulation to be delivered and discarded once it has reached its maximal usage. Alternatively, the dry powder inhaler is reusable and is provided with a replaceable cartridge for single use to deliver a single dose using a single inhalation provided by a subject. In this embodiment, multiple cartridges of a specific powder content containing an active ingredient and packaged, for example, in a blister pack can be provided with a single inhaler for multiple uses by a subject. In this and other embodiments, a cartridge can comprise a thy powder formulation for treating a variety of conditions, diseases or disorders.

A system for the delivery of an inhalable dry powder is also provided, comprising: a) a dry powder comprising a medicament, and b) an inhaler comprising a powder containing cartridge, the cartridge comprising a gas inlet and a gas outlet, and a housing in which to mount the cartridge and defining two flow pathways, a first flow pathway allowing gas to enter the gas inlet of the cartridge, a second flow pathway allowing gas to bypass the enclosure gas inlet, and a mouthpiece and upon applying a pressure drop of ≥2 kPa across the inhaler plume of particles is emitted from the mouthpiece wherein 50% of said emitted particles have a VMAD of ≤10 μm, wherein flow bypassing the cartridge gas inlet is directed to impinge upon the flow exiting the enclosure substantially perpendicular to the gas outlet flow direction.

An inhalation system for delivering a dry powder formulation to a patient's lung(s) is provided, the system comprising a dry powder inhaler configured to have flow conduits with a total resistance to flow in a dosing configuration ranging in value from 0.065 to about 0.200 (kPa)/liter per minute.

### III. Powder Formulations and Methods of Making the Same

These present devices and systems are useful in pulmonary delivery of powders with a wide range of characteristics. Embodiments include systems comprising an inhaler, an integral or installable unit dose cartridge comprising the desirable powder doses. Pulmonary delivery of powders includes carriers and excipients which safety and efficacy have been proven in commercially available products. Dry powders be made by lyophilizing, or spray-drying solution or suspensions of the various desired formulations. Crystalline microparticles with a specific surface area (SSA) of between about 35 and about 67 m²/g exhibit characteristics beneficial to delivery of drugs to the lungs such as improved aerodynamic performance and improved drug adsorption. In some embodiments, high capacity crystalline microparticles have a specific surface area which is less than 35 m²/g and specific surface area of these particles can range from about 19 m²/g to about 30 m²/g or from about 28 m²/g to about 71 m²/g, or from about 19 m²/g to about 57 m²/g depending on the amount of active agent. In some embodiments, microparticles can have specific surface area ranging from about 4 m²/g to about 30 m²/g and have improved aerodynamic properties as measured by flyability and flowability.

In one embodiment, the dry powder medicament may forms particles, microparticles and the like, which can be used as carrier systems for the delivery of active agents to a target site in the body. The term “active agent” is referred to herein as the therapeutic agent that can be encapsulated, associated, joined, complexed or entrapped within or adsorbed onto the diketopiperazine formulation. The dry powder medicament can be used to deliver biologically active agents having therapeutic, prophylactic or diagnostic activities.

Microparticles for pulmonary delivery having a diameter of between about 0.5 and about 10 μm can reach the lungs and can reach the systemic circulation and deliver an active agent. A diameter of less than about 10 μm is required to navigate the turn of the throat and a diameter of about 0.5 μm or greater is required to avoid being exhaled. Generally, microparticles having diameters greater than 10 μm or greater than 20 μm are useful for local delivery to the respiratory tract and lungs.

Microparticles having a diameter of between about 0.5 and about 10 microns can reach the lungs, successfully passing most of the natural barriers. A diameter of less than about 10 microns is required to navigate the turn of the throat and a diameter of about 0.5 microns or greater is required to avoid being exhaled. Microparticles with a specific surface area (SSA) of between about 4 and about 71 m²/g may exhibit characteristics beneficial to delivery of drugs to the lungs such as improved aerodynamic performance and improved drug adsorption.

In certain embodiments, a composition for pulmonary delivery is provided with an active agent comprises a plurality of substantially uniformly formed, microcrystalline particles, wherein the particles have a substantially hollow spherical structure and comprise a shell that do not self-assemble, and the particles have a volumetric mean geometric diameter less than equal to 5 μm; wherein the particles are formed by a method comprising the step of combining an excipient in a solution without the presence of a surfactant and concurrently homogenizing in a high shear mixer at high pressures of up to 2,000 psi to form a precipitate; washing the precipitate in suspension with deionized water; concentrating the suspension and drying the suspension in a spray drying apparatus.

The microcrystalline particles can have a substantially hollow spherical structure and comprise a shell which can be porous. In certain embodiments, the microcrystalline particles can be substantially hollow spherical and substantially solid particles comprising the drug and/or drug content provided and other factors in the process of making the powders. In one embodiment, the microcrystalline particles comprise particles that are relatively porous, having average pore volumes of about 0.43 cm³/g, ranging from about 0.4 cm³/g to about 0.45 cm³/g, and average pore size ranging from about 23 nm to about 30 nm, or from about 23.8 nm to 26.2 nm as determined by BJH adsorption.

Certain embodiments disclosed herein comprise powders comprising a plurality of substantially uniform, microcrystalline particles, wherein the particles have a substantially spherical structure comprising a shell which can be porous, and the particles comprise crystallites of a diketopiperazine that do not self-assemble in suspension or solution and have a volumetric median geometric diameter less than 5 μm; or less than 2.5 μm.

In a particular embodiment herein, up to about 92% of the microcrystalline particles have a volumetric median geometric diameter of 5.8 μm. In one embodiment, the particle's shell is constructed from interlocking diketopiperazine microcrystals having one or more drugs adsorbed on their surfaces. In some embodiments, the particles can entrap the drug in their interior void volume and/or combinations of the drug adsorbed to the crystallites' surface and drug entrapped in the interior void volume of the spheres.

The method can further comprise the steps of adding with mixing a solution comprising an active agent or an active ingredient such as a drug or bioactive agent prior to the spray drying step so that the active agent or active ingredient is adsorbed and/or entrapped on or within the particles. Particles made by this process can be in the submicron size range prior to spray-drying.

In certain embodiments where the starting material comprising the active ingredient is an extract exhibiting a high degree of viscocity, or a substance having a honey like viscous appearance, the microcrystalline particles are formed as above and by washing them in water using tangential flow filtration prior to combining with the extract or viscous material. After washing in water, the resultant particle suspension is lyophilized to remove the water and re-suspended in an alcohol solution, including ethanol or methanol prior to adding the active ingredient as a solid, or in a suspension, or in solution. In one embodiment, optionally, the method of making the composition comprises the step of adding any additional excipient, including one or more, amino acid, such as leucine, isoleucine, norleucine, methionine or one or more phospholipids, for example, 1,2-dipalmitoyl-sn-glycero-3-phosphocholine (DPPC) or 1,2-distearoyl-sn-glycero-3-phosphocholine (DSPC), concurrently with the active ingredient or subsequent to adding the active ingredient, and prior to spray drying. In certain embodiments, Formation of the composition comprises the step wherein the extract comprising desired active agents is optionally filtered or winterized to separate and remove layers of unwanted materials such as lipids to increase its solubility.

The method can further comprise the steps of adding with mixing a solution, the mixing can optionally be performed with or without homogenization in a high shear mixer, the solution comprising an active agent or an active ingredient such as a drug or bioactive agent prior to the spray drying step so that the active agent or active ingredient is adsorbed and/or entrapped on or within the particles. Particles made by this process can be in the submicron size range prior to spray-drying, or the particles can be formed from the solution during spray-drying.

In some embodiments herewith, the drug content can be delivered at about 0.01% (w/w) of the powder formulation. In one embodiment, the drug content to be delivered from about 0.01% (w/w) to about 75% (w/w); from about 1% to about 50% (w/w), from about 10% (w/w) to about 25% (w/w), or from about 10% to about 20% (w/w), or from 5% to about 30%, or greater than 25%.

In alternate embodiments, the pharmaceutically acceptable carrier for making dry powders can comprise any carriers or excipients useful for making dry powders and which are suitable for pulmonary delivery. Example of suitable carriers and excipients include, sugars, including saccharides and polysaccharides, such as lactose, mannose, sucrose, mannitol, trehalose; citrates, amino acids such as glycine, L-leucine, isoleucine, trileucine, tartrates, methionine, vitamin A, vitamin E, zinc citrate, trisodium citrate, zinc chloride, polyvinylpyrrolidone, polysorbate 80, phospholipids including diphosphotidylcholine and the like.

### IV. Cannabis Active Agents

The active agents for use in the compositions and methods described herein include a cannabis agent. These can include both plant-based and synthetic materials.

1. Plant-Based Materials

Cannabis is a genus of flowering plants in the family Cannabaceae. The number of species within the genus is disputed. Three species may be recognized: Cannabis sativa, Cannabis indica, and Cannabis ruderalis; C. ruderalis may be included within C. sativa; all three may be treated as subspecies of a single species, C. sativa or C. sativa may be accepted as a single undivided species. The genus is widely accepted as being indigenous to and originating from Central Asia, with some researchers also including upper South Asia in its origin.

The plant is also known as hemp, although this term is often used to refer only to varieties of Cannabis cultivated for non-drug use. Cannabis has long been used for hemp fibre, hemp seeds and their oils, hemp leaves for use as vegetables and as juice, medicinal purposes, and as a recreational drug. Industrial hemp products are made from cannabis plants selected to produce an abundance of fiber. To satisfy the UN Narcotics Convention, some cannabis strains have been bred to produce minimal levels of tetrahydrocannabinol (THC), the principal psychoactive constituent. Some strains have been selectively bred to produce a maximum of THC (a cannabinoid), the strength of which is enhanced by curing the flowers. Various compounds, including hashish and hash oil, are extracted from the plant.

Globally, in 2013, 60,400 kilograms of cannabis were produced legally. In 2014 there were an estimated 182.5 million cannabis users (3.8% of the population aged 15-64). This percentage has not changed significantly between 1998 and 2014. Cannabis can be used by smoking, vaporizing, within food, or as an extract.

Medical cannabis (or medical marijuana) refers to the use of cannabis and its constituent cannabinoids, to treat disease or improve symptoms. Medical cannabis use takes place in Canada, Belgium, Australia, the Netherlands, Germany, Spain, and 31 U.S. states. In September 2018, cannabis was legalized in South Africa while Canada legalized recreational use of cannabis in October 2018.

Cannabis is used to reduce nausea and vomiting during chemotherapy, to improve appetite in people with HIV/AIDS and to treat chronic pain and muscle spasms. Cannabinoids are under preliminary research for their potential to affect stroke. Short-term use increases both minor and major adverse effects. Common side effects include dizziness, feeling tired, vomiting, and hallucinations. Long-term effects of cannabis are not clear. Concerns including memory and cognition problems, risk of addiction, schizophrenia in young people, and the risk of children taking it by accident.

The main psychoactive part of cannabis is tetrahydrocannabinol (THC), one of 483 known compounds in the plant, including at least 65 other cannabinoids. Cannabis has mental and physical effects, such as creating a “high” or “stoned” feeling, a general change in perception, heightened mood, and an increase in appetite. Onset of effects is within minutes when smoked, and about 30 to 60 minutes when cooked and eaten. They last for between two and six hours. The high lipid-solubility of cannabinoids results in their persisting in the body for long periods of time. Even after a single administration of THC, detectable levels of THC can be found in the body for weeks or longer (depending on the amount administered and the sensitivity of the assessment method). A number of investigators have suggested that this is an important factor in marijuana's effects, perhaps because cannabinoids may accumulate in the body, particularly in the lipid membranes of neurons.

Researchers have confirmed that THC exerts its most prominent effects via its actions on two types of cannabinoid receptors, the CB₁ receptor and the CB₂ receptor, both of which are G protein-coupled receptors. The CB₁ receptor is found primarily in the brain as well as in some peripheral tissues, and the CB₂ receptor is found primarily in peripheral tissues but is also expressed in neuroglial cells. THC appears to alter mood and cognition through its agonist actions on the CB₁ receptors, which inhibit a secondary messenger system (adenylate cyclase) in a dose-dependent manner. These actions can be blocked by the selective CB₁ receptor antagonist rimonabant (SR141716), which has been shown in clinical trials to be an effective treatment for smoking cessation, weight loss, and as a means of controlling or reducing metabolic syndrome risk factors. However, due to the dysphoric effect of CB₁ receptor antagonists, this drug is often discontinued due to these side effects.

Via CB₁ receptor activation, THC indirectly increases dopamine release and produces psychotropic effects. Cannabidiol (CBD) also acts as an allosteric modulator of the μ- and δ-opioid receptors. THC also potentiates the effects of the glycine receptors. It is unknown if or how these actions contribute to the effects of cannabis. CBD is a 5-HT_{1A} receptor agonist, which may also contribute to an anxiolytic effect. This likely means the high concentrations of CBD found in Cannabis indica mitigate the anxiogenic effect of THC significantly. The cannabis industry claims that sativa strains provide a more stimulating psychoactive high while indica, strains are more sedating with a body high; however, this is disputed by researchers.

2. Synthetic Materials

Synthetic cannabinoids are a class of molecules that bind to cannabinoid receptors in the body (the same receptors to which THC and CBD attach, which are cannabinoids in cannabis plants). They are designer drugs that are commonly sprayed onto plant matter and are usually smoked, although since 2016 they have also been consumed in a concentrated liquid form in the US and UK. They have been marketed as herbal incense, or “herbal smoking blends.” They are often labeled “not for human consumption” for liability defense.

When the herbal blends went on sale in the early 2000s, it was thought that they achieved psychoactive effects from a mixture of natural herbs. Laboratory analysis in 2008 showed instead that many contained synthetic cannabinoids. Since 2016 synthetic cannabinoids are the most common new psychoactive substances to be reported. From 2008 to 2014, 142 synthetic cannabinoids were reported to the European Monitoring Centre for Drugs and Drug Addiction (EMCDDA). A large and complex variety of synthetic cannabinoids are designed in an attempt to avoid legal restrictions on cannabis, making synthetic cannabinoids designer drugs.

Most synthetic cannabinoids are agonists of the cannabinoid receptors. They have been designed to be similar to THC, the natural cannabinoid with the strongest binding affinity to the CB₁ receptor, which is linked to the psychoactive effects or “high” of marijuana. These synthetic analogs often have greater binding affinity and greater potency to the CB₁ receptors. There are several synthetic cannabinoid families (e.g., CP-xxx, WIN-xxx, JWH-xxx, UR-xxx, and PB-xx) classified based on the base structure.

Reported user negative effects include palpitations, paranoia, intense anxiety, nausea, vomiting, confusion, poor coordination, and seizures. There have also been reports of a strong compulsion to re-dose, withdrawal symptoms, and persistent cravings. There have been several deaths linked to synthetic cannabinoids. The Centers for Disease Control and Prevention (CDC) found that the number of deaths from synthetic cannabinoid use tripled between 2014 and 2015.

Use of the term “synthetic marijuana” to describe products containing synthetic cannabinoids is controversial and a misnomer. Relative to marijuana, is has been argued that products containing synthetic cannabinoids are quite different, and the effects are much more unpredictable. Since the term synthetic does not apply to the plant, but rather to the cannabinoid that the plant contains (THC), the term synthetic cannabinoid is more appropriate.

Synthetic cannabinoids were made for cannabinoid research focusing on tetrahydrocannabinol (THC), the main psychoactive and analgesic compound found in the cannabis plant. Synthetic cannabinoids were needed partly due to legal restrictions on natural cannabinoids, which make them difficult to obtain for research. Tritium-labelled cannabinoids such as CP-55,940 were instrumental in discovering the cannabinoid receptors in the early 1990s.

Some early synthetic cannabinoids were also used clinically. Nabilone, a first-generation synthetic THC analog, has been used as an antiemetic to combat vomiting and nausea, since 1981. Synthetic THC (marinol, dronabinol) has been used as an antiemetic since 1985 and an appetite stimulant since 1991.

In the early 2000s, synthetic cannabinoids began to be used for recreational drug use in an attempt to get similar effects to cannabis. Because synthetic cannabinoid molecular structures differ from THC and other illegal cannabinoids, synthetic cannabinoids were not technically illegal. Since the discovery of the use of synthetic cannabinoids for recreational use in 2008, some synthetic cannabinoids have been made illegal, but new analogs are continually synthesized to avoid the restrictions. Synthetic cannabinoids have also been used recreationally because they are inexpensive and are typically not revealed by the standard marijuana drug tests. Unlike nabilone, the synthetic cannabinoids found being used for recreational use did not have any documented therapeutic effects.

There are five major categories for synthetic cannabinoids: classical cannabinoids, non-classical cannabinoids, hybrid cannabinoids, aminoalkylindoles, and eicosanoids. Classical cannabinoids are analogs of THC that are based on a dibenzopyran ring. They were developed starting in the 1960s, following the isolation of THC, and were originally the only cannabinoids synthesized. Classical cannabinoids include nabilone and dronabinol, and one of the best known synthetic classical cannabinoids is HU-210. HU-210 is a chiral compound first synthesized by Raphael Mechoulam at Hebrew University in the 1980s.

Non-classical cannabinoids include cyclohexylphenols (CP), which were first synthesized in the late 1970s to 1980s by Pfizer as potential analgesics. The C8 homologue of CP-47,497 (CP-47,497-C8) was one of the first synthetic cannabinoids being used recreationally. CP-47,497-C8 is made by extending the dimethylheptyl side chain of CP-47,497 to a dimethyloctyl side chain. It was discovered by forensic scientists in an herbal blend known as “Spice” in 2008, along with JWH-018, an aminoalkylindole.

Hybrid cannabinoids have a combination of classical and non-classical cannabinoid structural features. For example, AM-4030, a derivative of HU-210, is a hybrid cannabinoid because it has the dibenzopyran ring common of classical cannabinoids and an aliphatic hydroxyl group common in the CP family of nonclassical cannabinoids.

Aminoalkylindoles are structurally dissimilar to THC and include naphthoylindoles (JWH-018), phenyl acetylindoles (JWH-250), and benzoylindoles (AM-2233). Aminoalkylindoles are considered to be the most common synthetic cannabinoids found in synthetic cannabinoid blends, likely due to the fact that these molecules are easier to synthesize than classical and non-classical cannabinoids. The JWH molecules were first synthesized by Professor John William Huffman at Clemson University in the late 1990s. The FBI concluded in a 2012 memo that as a result of the publication of J. W. Huffman's research, people searching for a “marijuana-like-high” would follow his recipes and methods.

Eicosanoid synthetic cannabinoids are analogs of endocannabinoids, such as anandamide. Endocannabinoids are cannabinoids naturally occurring in the body. One of the best-known synthetic analogs of anandamide is methanandamide.

The synthetic cannabinoids that have emerged recently have even greater structural diversity, possibly to subvert legal regulations on earlier generations of synthetic cannabinoids. The indazole carboxamide group, including APINACA (AKB-48), an adamantyl indazole carboxamide, and AB-PINACA, an aminocarbonyl indazole carboxamide, is an example of a new group of synthetic cannabinoids. Most clandestine manufacturers and producers only make small changes to the structure of a synthetic cannabinoid, such as changing an indole to indazole structure (AM-2201 to THJ-2201) or terminal fluorine replacement; however, one group that was unprecedented when discovered by forensic scientists in 2013, was the quinolinyl ester synthetic cannabinoids.

PB-22 and 5F-PB-22 were the first synthetic cannabinoids to include a quinoline substructure and an ester linkage. These compounds are thought to have been synthesized with the intention of making a synthetic cannabinoid prodrug, which might improve absorption and confound detection. Ester bonds are easily biodegradable through spontaneous or endogenous, nonspecific esterase hydrolysis, which has been commonly used in medicinal chemistry to make ester prodrugs.

Although most synthetic cannabinoids are not direct analogs of THC, they share many common features with THC. Most are lipid-soluble, non-polar, small molecules (usually 20-26 carbon atoms) that are fairly volatile, making them “smokable,” like THC. Another common feature of most synthetic cannabinoids and THC is a side-chain of 5-9 saturated carbon atoms. It has been found that this chain of 5-9 carbons is required for optimal psychotropic activity from binding CB₁ receptors. Also, most synthetic cannabinoids are agonists of both cannabinoid receptors, CB₁ and CB₂, like THC; however, they often have greater binding affinity and therefore greater potency than THC, as seen in Table 2. Due to the greater potency, the standard doses of many synthetic cannabinoids may be less than 1 mg.

### V. Treating Diseases and Disorders

The method of treatment comprises providing to a patient in need of treatment a dry powder inhaler comprising a cartridge containing a dose of an inhalable formulation comprising a cannabis agent and a pharmaceutical acceptable carrier and/or excipient; and having the patient inhale through the dry powder inhaler deeply for about 3 to 4 seconds to deliver the dose. In the method, the patient can resume normal breathing pattern thereafter.

Medical cannabis has several potential beneficial effects. Evidence is moderate that it helps in chronic pain and muscle spasms. Other evidence suggests its use for reducing nausea during chemotherapy, improving appetite in HIV/AIDS, improving sleep, and improving tics in Tourette syndrome. When usual treatments are ineffective, cannabinoids have also been recommended for anorexia, arthritis, migraine, and glaucoma. It is recommended that cannabis use be stopped in pregnancy.

1. Nausea

Medical cannabis is somewhat effective in chemotherapy-induced nausea and vomiting (CINV) and may be a reasonable option in those who do not improve following preferential treatment. Comparative studies have found cannabinoids to be more effective than some conventional antiemetics such as prochlorperazine, promethazine, and metoclopramide in controlling CINV, but these are used less frequently because of side effects including dizziness, dysphoria, and hallucinations. Long-term cannabis use may cause nausea and vomiting, a condition known as cannabinoid hyperemesis syndrome.

A 2016 Cochrane review said that cannabinoids were “probably effective” in treating chemotherapy-induced nausea in children, but with a high side-effect profile (mainly drowsiness, dizziness, altered moods, and increased appetite). Less common side effects were ocular problems, orthostatic hypotension, muscle twitching, pruritis, vagueness, hallucinations, lightheadedness and dry mouth.

2. HIV/AIDS

Evidence is lacking for both efficacy and safety of cannabis and cannabinoids in treating patients with HIV/AIDS or for anorexia associated with AIDS. As of 2013, current studies suffer from effects of bias, small sample size, and lack of long-term data.

3. Pain

A 2017 review found only limited evidence for the effectiveness of cannabis in relieving chronic pain in several conditions. Another review found tentative evidence for use of cannabis in treating peripheral neuropathy, but little evidence of benefit for other types of long term pain.

When cannabis is inhaled to relieve pain, blood levels of cannabinoids rise faster than when oral products are used, peaking within three minutes and attaining an analgesic effect in seven minutes. A 2014 review found limited and weak evidence that smoked cannabis was effective for chronic non-cancer pain. A 2015 meta-analysis found that inhaled medical cannabis was effective in reducing neuropathic pain in the short term for one in five to six patients. Another 2015 review found limited evidence that medical cannabis was effective for neuropathic pain when combined with traditional analgesics. A 2011 review considered cannabis to be generally safe, and it appears safer than opioids in palliative care.

4. Neurological Problems

Cannabis' efficacy is not clear in treating neurological problems, including multiple sclerosis (MS), epilepsy, and movement problems. The combination of Δ9-tetrahydrocannabinol (THC) and cannabidiol (CBD) extracts give subjective relief of spasticity, though objective post-treatment assessments do not reveal significant changes. Evidence also suggests that oral cannabis extract is effective for reducing patient-centered measures of spasticity. A trial of cannabis is deemed to be a reasonable option if other treatments have not been effective. Its use for MS is approved in ten countries. A 2012 review found no problems with tolerance, abuse, or addiction.

5. Epilepsy

Epilepsy (also called epileptic seizure disorder) is a chronic brain disorder characterized by recurrent (≥2) seizures that are unprovoked (ie, not related to reversible stressors) and that occur >24 h apart. A single seizure is not considered an epileptic seizure. Epilepsy is often idiopathic, but various brain disorders, such as malformations, strokes, and tumors, can cause symptomatic epilepsy.

6. Dravet Syndrome

Dravet syndrome is a severe infantile-onset, genetic, drug-resistant epilepsy syndrome with a distinctive but complex electroclinical presentation. Onset of Dravet syndrome occurs during the first year of life with clonic seizures (jerking) and tonic-clonic (convulsive) seizures in previously healthy and developmentally normal infants. Symptoms peak at about five months of age, and the latest onset beginning by 15 months of age. Other seizures develop between one and four years of age such as prolonged focal dyscognitive seizures and brief absence seizures, and duration of these seizures decreases during this period, but their frequency increases. Prognosis is poor, with death occurring in approximately 14 percent of children. Death can be caused by the seizures themselves, by infection due to prolonged periods of physical inactivity, or by the presence of advanced neurodegenerative disease or a compromised level of consciousness requiring a feeding tube. Death can also occur suddenly due to uncertain causes, often because of the relentless neurological decline or from Sudden Unexpected Death in Epilepsy.

7. Lennox-Gastaut Syndrome (LGS)

LGS is a type of epilepsy with multiple types of seizures, particularly tonic (stiffening) and atonic (drop) seizures. According to Trevathan et al. in the December 1997 edition of Epilepsia, the estimated prevalence of LGS is between 3 percent and 4 percent of childhood epilepsy cases. LGS affects between 14,500 to 18,500 children under the age of 18 years in the U.S. and over 30,000 children and adults in the U.S. Eighty percent of children with LGS continue to experience seizures, psychiatric, intellectual and behavioral deficits in adulthood. Seizures due to LGS are hard to control and generally require life-long treatment.

8. Tuberous Sclerosis Complex

Tuberous sclerosis complex (TSC) is a neurocutaneous syndrome that occurs in 1 of 6000 children; 85% of cases involve mutations in the TSC1 gene (9q34), which controls the production of hamartin, or the TSC2 gene (16p13.3), which controls the production of tuberin. These proteins act as growth suppressors If either parent has the disorder, children have a 50% risk of having it. However, new mutations account for two thirds of cases. Patients with TSC have tumors or abnormalities that manifest at different ages and in multiple organs, including the brain, heart, eyes, kidneys, lungs and skin

9. Rett Syndrome

Rett syndrome (RTT) is a rare, non-inherited, X-linked neurodevelopmental disorder affecting approximately 1 in 10,000 to 15,000 live female births. RTT is most commonly caused by heterozygous de-novo mutations in the gene encoding methyl-CpG-binding protein 2 (MeCP2) resulting in a loss of function of the MeCP2 protein. The condition affects predominantly females and it results in abnormal neuronal development and function in affected children. The symptomatology of RTT is progressive, with early onset from about 6-18 months of life, followed by a rapid destructive phase at the age of 1 to 4 years. This stage is characterized by loss of purposeful hand skills, loss of spoken language, breathing and cardiac irregularities, microcephaly, and autistic-like behaviors. After the period of regression, patients enter a prolonged period of stabilization where most of the impairments associated with the destructive phase persist together with apraxia, motor problems, and seizures. Over time, the patient's motor function continues to deteriorate, resulting in reduced mobility, scoliosis, rigidity, muscular weakness and spasticity.

10. Autism Spectrum Disorders

Autism spectrum disorders are neurodevelopmental disorders characterized by impaired social interaction and communication, repetitive and stereotyped patterns of behavior, and uneven intellectual development often with intellectual disability. Symptoms begin in early childhood. The cause in most children is unknown, although evidence supports a genetic component, in some patients, the disorders may be caused by a medical condition. Diagnosis is based on developmental history and observation. Treatment consists of behavioral management and sometimes drug therapy. Autism spectrum disorders represent a range of neurodevelopmental differences that are considered neurodevelopmental disorders.

11. Posttraumatic Stress Disorder

There is tentative evidence that medical cannabis is effective at reducing posttraumatic stress disorder symptoms, but, as of 2017, there is insufficient evidence to confirm its effectiveness for this condition.

12. Other Conditions

Other conditions that may be treated using the cannabis dry powder formulations disclosed herein include for treating neurodermitis, contact eczema, allergies, for the prevention or treatment of phototoxic reactions, for the treatment of conglobata, itching dermatoses, rosacea, perioral dermatitis, acne, acne conglobata, psoriasis (vulgaris, arthropathica, pustulosa), mosquito bites, skin atrophy (in particular also cortisone-related skin changes), allergic rhinitis, privinismus, conjunctivitis, otitis externa, bronchial asthma, COPD, Crohn's disease, ulcerative colitis, sarcoidosis, or inflammatory-rheumatic diseases of the soft tissue or joints, or external mycoses.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents that are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

What is claimed:

1. A dry powder inhaler comprising a dry powder composition of (a) a plant-based cannabidiol (pCBD) composition; (b) a plant-based tetrahydrocannabinol composition (pTHC); (c) a synthetic cannabidiol composition (sCBD); (d) a synthetic tetrahydrocannabinol composition (sTHC); (e) a plant-based cannabidiol (pCBD) composition in combination with a plant-based tetrahydrocannabinol composition (pTHC); or (f) a synthetic cannabidiol composition (sCBD) in combination with a synthetic tetrahydrocannabinol composition (sTHC), wherein the dry powder composition is produced by thin film freezing.

2. The dry powder inhaler of claim 1, wherein the dry powder composition is a pharmaceutical composition for inhalation.

5. A method of delivering a dry powder composition to the airway of a subject comprising administering said dry powder composition to an airway of said subject with a dry powder inhaler wherein the dry powder composition comprises: (a) a plant-based cannabidiol (pCBD) composition; (b) a plant-based tetrahydrocannabinol composition (pTHC); (c) a synthetic cannabidiol composition (sCBD); (d) a synthetic tetrahydrocannabinol composition (sTHC); (e) a plant-based cannabidiol (pCBD) composition in combination with a plant-based tetrahydrocannabinol composition (pTHC); or (f) a synthetic cannabidiol composition (sCBD) in combination with a synthetic tetrahydrocannabinol composition (sTHC), wherein the dry powder composition is produced by thin film freezing.

3. A method for treating a subject comprising administering an effective amount of an inhaled dry powder composition comprising: (a) a plant-based cannabidiol (pCBD) composition; (b) a plant-based tetrahydrocannabinol composition (pTHC); (c) a synthetic cannabidiol composition (sCBD); (d) a synthetic tetrahydrocannabinol composition (sTHC); (e) a plant-based cannabidiol (pCBD) composition in combination with a plant-based tetrahydrocannabinol composition (pTHC); or (f) a synthetic cannabidiol composition (sCBD) in combination with a synthetic tetrahydrocannabinol composition (sTHC), wherein the dry powder composition is produced by thin film freezing.

4. The method of claim 3, wherein the subject has a pulmonary disease.

5. The method of claim 4, wherein the pulmonary disease is COPD or asthma.

6. The method of claim 3, wherein the subject has a neurological disease or disorder.

7. The method of claim 6, wherein the neurological disease or disorder comprises Alzheimer's disease, epilepsy, an autism spectrum disorder, PTSD, Parkinson's disease, Huntington's disease, stroke, major depression or traumatic brain injury.

8. The method of claim 3, wherein the subject has ocular disease.

9. The method of claim 8, wherein the subject has macular degeneration, glaucoma or retinitis pigmentosa (RP).

10. The method of claim 3, wherein the subject has Rett syndrome (RTT), Lennox-Gastaut Syndrome (LGS), Tuberous Sclerosis Complex (TSC), Dravet syndrome, nausea or HIV infection.

11. A dry powder inhaler comprising a dry powder composition of (a) a plant-based cannabidiol (pCBD) composition in combination with a plant-based tetrahydrocannabinol composition (pTHC); or (b) a synthetic cannabidiol composition (sCBD) in combination with a synthetic tetrahydrocannabinol composition (sTHC).

12. The dry powder inhaler of claim 11, wherein the dry powder composition is a pharmaceutical composition for inhalation.

3. The dry powder inhaler of claim 11, wherein the dry powder composition comprises a pCBD:pTHC in a ratio of 10:1, 5:1, 2:1, 1:1, 1:2, 1:5 or 1:10.

4. The dry powder inhaler of claim 11, wherein the dry powder composition comprises a sCBD:sTHC in a ratio of 10:1, 5:1, 2:1, 1:1, 1:2, 1:5 or 1:10.

5. A method of delivering a dry powder composition of (a) a plant-based cannabidiol (pCBD) composition in combination with a plant-based tetrahydrocannabinol composition (pTHC); or (b) a synthetic cannabidiol composition (sCBD) in combination with a synthetic tetrahydrocannabinol composition (sTHC) to the airway of a subject comprising administering said dry powder composition to an airway of said subject with a dry powder inhaler.

13. A method for treating a subject comprising administering an effective amount of an inhaled dry powder composition comprising: (a) a plant-based cannabidiol (pCBD) composition in combination with a plant-based tetrahydrocannabinol composition (pTHC); or (b) a synthetic cannabidiol composition (sCBD) in combination with a synthetic tetrahydrocannabinol composition (sTHC).

14. The method of claim 13, wherein the subject has a pulmonary disease.

15. The method of claim 14, wherein the pulmonary disease is COPD or asthma.

16. The method of claim 13, wherein the subject has a neurological disease or disorder.

17. The method of claim 16, wherein the neurological disease or disorder comprises Alzheimer's disease, epilepsy, an autism spectrum disorder, PTSD, Parkinson's disease, Huntington's disease, stroke, major depression or traumatic brain injury.

18. The method of claim 13, wherein the subject has ocular disease.

19. The method of claim 18, wherein the subject has macular degeneration, glaucoma or retinitis pigmentosa (RP).

20. The method of claim 13, wherein the subject has Rett syndrome (RTT), Lennox-Gastaut Syndrome (LGS), Tuberous Sclerosis Complex (TSC), Dravet syndrome, nausea or HIV infection.
